(19) United States
(12) Patent Application Publication
(10) Pub. No.: US 2026/0115191 A1
(43) Pub. Date: Apr. 30, 2026

(54) Nilotinib Compositions with Enhanced Solubility

(71) Applicant: Neoform Biopharmaceutical Limited, Shenzhen (CN)

(72) Inventors: Stephen R. Byrn, West Lafayette, IN (US); Dan Smith, West Lafayette, IN (US); Anne Zimmermann, Shenzhen (CN); Bjarke Strøm Larsen, Oslo (NO); Fang Tian, Shenzhen (CN)

(21) Appl. No.: 19/201,221

(22) Filed: May 7, 2025

Related U.S. Application Data
(63) Continuation of application No. 17/178,485, filed on Feb. 18, 2021, now abandoned.
(60) Provisional application No. 62/979,101, filed on Feb. 20, 2020.

(51) Int. Cl. A61K 31/506; A61K 9/14; A61K 47/38; A61K 47/69

(52) U.S. Cl. CPC A61K 31/506; A61K 9/146; A61K 47/38; A61K 47/6921

(57) ABSTRACT

Disclosed herein are compositions that contain a salt of Nilotinib and a polymer, which composition have improved properties in terms of solubility and bioavailability and which are useful for treating disorders of uncontrolled cellular proliferation. Methods of using the compositions are also described

### CROSS-REFERENCE TO RELATED APPLICATIONS

This application is a continuation of U.S. application Ser. No. 17/178,485, filed on Feb. 18, 2021, which claims the benefit of U.S. Provisional Application No. 62/979,101, filed on Feb. 20, 2020, the contents of which are incorporated herein by reference in their entireties.

### BACKGROUND

Nilotinib, having the chemical name 4-methyl-N-[3-(4-methylimidazol-1-yl)-5-(trifluoromethyl)phenyl]-3-[(4-pyridin-3-ylpyrimidin-2-yl)amino]benzamide, is a pharmaceutically active compound used for the treatment of Philadelphia-chromosome-positive chronic myelogenous leukemia. It belongs to the therapeutic group of tyrosine kinase inhibitors, a group which has revolutionized the treatment of cancers, as they act much more specifically toward malignant cells in comparison to conventional cytotoxic chemotherapy. However, Nilotinib hydrochloride, which is the commercial salt used in marketed products can exist in a number of different solid forms including anhydrates, hydrates and solvates, all of which exhibit poor aqueous solubility.

There is a continuing need for the development of improved oral solid dosage forms for tyrosine kinase inhibitors, such as Nilotinib, which have suitable dissolution profile and stability and which do not necessitate high vehicle volumes.

Thus, there is a need in the art to increase the solubility and bioavailability of Nilotinib without compromising the physical stability of Nilotinib. Such compositions and methods are disclosed herein.

### SUMMARY

In accordance with the purpose(s) of the invention, as embodied and broadly described herein, the invention, in one aspect, relates to compositions and methods of treating disorders of uncontrolled cellular proliferation.

Disclosed herein is a composition comprising: a) Nilotinib, salified with b) a polymer selected from the group consisting of hydroxypropylmethyl-cellulose acetate succinate (HPMCAS), hydroxypropylmethylcellulose phthalate (HPMCP), cellulose acetate trimellitate (CAT), cellulose acetate phthalate (AP), hydroxypropylcellulose acetate phthalate (HPCAP), hydroxypropylmethylcellulose acetate phthalate (HPMCAP), and methylcellulose acetate phthalate (MCAP); wherein the composition exhibits, in a dissolution experiment, at least a 1.25-fold improvement in solubility within the first 2 hours over a control composition comprising Nilotinib, not being salified with the polymer as measured according to a dissolution profile where FaSSIF is used as a medium and having a pH of 6.5.

Also disclosed is a composition of Nilotinib and a polymer wherein said solid dosage form releases at least 20% of the Nilotinib in 90 min in a dissolution study in FaSSIF in a dissolution bath at a pH of 6.5, utilizing a stirring rate of 100 rpm wherein the weight ratio of the amount of drug in said dosage form to the water is about 1/9.

Also disclosed is a composition of Nilotinib salified with HPMCP wherein an infrared spectrum of the composition does not have a N—H deformation peak at 1498 cm⁻¹ and does have a peak at about 1691 cm⁻¹.

Also disclosed is a method for treating a disorder of uncontrolled cellular proliferation, the method comprising the step of administering to the subject an effective amount of a composition comprising: a) Nilotinib, salified with b) a polymer selected from the group consisting of hydroxypropylmethyl-cellulose acetate succinate (HPMCAS), hydroxypropylmethylcellulose phthalate (HPMCP), cellulose acetate trimellitate (CAT), cellulose acetate phthalate (CAP), hydroxypropylcellulose acetate phthalate (HPCAP), hydroxypropylmethylcellulose acetate phthalate (HPMCAP), and methylcellulose acetate phthalate (MCAP).

Also disclosed is a kit comprising an effective amount of a composition disclosed herein, and one or more of: (a) another tyrosine kinase inhibitor; (b) means for administering the composition; (c) instructions for use; (d) a drug for treatment of a disorder of uncontrolled cellular proliferation.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE DRAWINGS

The accompanying figures, which are incorporated in and constitute a part of this specification, illustrate several aspects and together with the description serve to explain the principles of the invention.

FIG. 1 shows an infrared spectrum of HPMCP (5), 40% Nilotinib loaded composition (4), 40% Nilotinib:HPMCP physical mixture (3), Nilotinib HCl salt (2) and Nilotinib freebase (1). The infrared spectra have been offset for clarity.

FIG. 2 shows dissolution measurements of Nilotinib containing capsule formulations equivalent to 40 mg NILOTINIB in 500 mL FaSSIF.

FIG. 3 shows dissolution measurements of capsules containing Nilotinib compositions in pH 1.0.

FIG. 4 shows an X-ray powder diffractogram of Nilotinib HPMCP salt measured immediately upon isolation by spray drying.

FIG. 5 shows particle size distribution of a Nilotinib HPMCP salt.

FIG. 6 shows in-vivo plasma concentration after administrating a single dose equivalent to 10 mg Nilotinib per kg body weight of rats.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### 1. Definitions

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or cannot occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. In one aspect, a subject can be a human. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects. In some aspects of the disclosed methods, the subject has been diagnosed with a need for treatment of one or more disorders prior to the administering step.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

A measure of the potential usefulness of an oral dosage form of a pharmaceutical agent is the dissolution profile observed after placing the dosage form in a standard USP dissolution apparatus. Various factors can affect the dissolution profile of a dosage form when placed in a dissolution bath. These factors include aqueous solubility, dissolution rate, solvent, stirring rate and dosage strength. Aqueous solubility is one of the most important of these factors. Tyrosine kinase inhibiting compounds typically are characterized by having poor aqueous solubility. The dissolution profile for some tyrosine kinase inhibitors, wherein the ratio of the amount of drug in said dosage form to dissolution medium (water) is about 1/9 (or 100 mg to 900 mL), indicate that the tyrosine kinase inhibitor is not substantially released and the release rate is low.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein. In some aspects of the disclosed methods, the subject has been diagnosed with a need for treatment of a viral infection prior to the administering step. As used herein, the phrase “identified to be in need of treatment for a disorder,” or the like, refers to selection of a subject based upon need for treatment of the disorder. It is contemplated that the identification can, in one aspect, be performed by a person different from the person making the diagnosis. It is also contemplated, in a further aspect, that the administration can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The term “contacting” as used herein refers to bringing a disclosed compound and a cell, target receptor, or other biological entity together in such a manner that the compound can affect the activity of the target (e.g., receptor, cell, etc.), either directly; i.e., by interacting with the target itself, or indirectly; i.e., by interacting with another molecule, co-factor, factor, or protein on which the activity of the target is dependent.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

The term “concentration of said basic dissolved drug or salt or either” is typically taken as referring to that material that either passes a 0.22 micron syringe filter or, alternatively, the material that remains in the supernatant following centrifugation of a sample. The filtration can be conducted using a pre-filter of pore size 2.0 μm, followed by filtration through a 0.22 μm PES syringe filter. The first 4-5 drops are used for equilibrating the filter, while the remaining sample can be used for analysis. Centrifugation can be typically carried out in a polypropylene microcentrifuge tube by centrifuging at 13,000 G for 60 seconds. It is recognized that this definition of “dissolved drug” encompasses not only monomeric solvated drug molecules but also a wide range of species that have submicron dimensions such as drug aggregates, aggregates of mixtures of polymer and drug, micelles, polymeric micelles, colloidal particles or nanocrystals, polymer/drug complexes, and other such drug-containing species that are present in the filtrate or supernatant in the: specified dissolution test.

As used herein, the term “use environment” generally means the gastrointestinal tract if in vivo and aqueous test medium if in vitro. More specifically, “use environment means” (1) if the use environment is in vivo and has a pH in the range of 1.0 to 2.0, the stomach; (2) if the use environment is in vivo and has a pH in the range of 5.0 to 7.0, the GI tract; (3) if the use environment is in vitro and has a pH in either of the ranges just mentioned, aqueous test fluid including FaSSIF which is initially at a pH of 1.0 to 2.0 and which can then be adjusted to within the range 5.0 to 7.0.

The term “solid dispersion” defines a system in a solid state (as opposed to a liquid or gaseous state) comprising at least two components, wherein one component is dispersed evenly throughout the other component or components. For example, an active ingredient or combination of active ingredients is dispersed in a matrix comprised of the pharmaceutically acceptable polymer(s) and pharmaceutically acceptable excipients. When said dispersion of the components is such that the system is chemically and physically uniform or homogenous throughout or consists of one phase (as defined in thermodynamics) or consists of a molecular mixture, such a solid dispersion will be called a “solid solution” or a “glassy solution”. A glassy solution is a homogeneous, glassy system in which a solute is dissolved in a glassy solvent. Glassy solutions and solid solutions of tyrosine kinase inhibitors are preferred physical systems. These systems do not contain any significant amounts of active ingredients in their crystalline or microcrystalline state, as evidenced by thermal analysis (DSC) or X-ray diffraction analysis including pair distribution function analysis. Solid solutions are preferred physical systems because the components therein readily form liquid solutions when contacted with a liquid medium such as gastric juice or water. The ease of dissolution may be attributed at least in part to the fact that the energy required for dissolution of the components from a solid solution is less than that required for the dissolution of the components from a crystalline or microcrystalline solid phase. This is because the crystalline phase is not present in a solid solution. In some cases the drug released from the solid solution may result in a high supersaturation and precipitate in the aqueous fluids of dissolution bath.

As used herein, “IC₅₀,” is intended to refer to the concentration of a substance (e.g., a compound or a drug) that is required for 50% inhibition of a biological process, or component of a process, including a protein, subunit, organelle, ribonucleoprotein, etc. In one aspect, an IC₅₀ can refer to the concentration of a substance that is required for 50% inhibition in vivo, as further defined elsewhere herein. In a further aspect, IC₅₀ refers to the half maximal (50%) inhibitory concentration (IC) of a substance.

As used herein, “EC₅₀,” is intended to refer to the concentration of a substance (e.g., a compound or a drug) that is required for 50% agonism of a biological process, or component of a process, including a protein, subunit, organelle, ribonucleoprotein, etc. In one aspect, an EC₅₀ can refer to the concentration of a substance that is required for 50% agonism in vivo, as further defined elsewhere herein. In a further aspect, EC₅₀ refers to the concentration of agonist that provokes a response halfway between the baseline and maximum response.

As used herein, “TCID₅₀,” is intended to refer to the concentration of a substance (e.g., a compound or a drug) that is required to produce infection, including a viral infection, in 50% of cell cultures that are inoculated.

As used herein, “EID₅₀,” is intended to refer to the concentration of a substance (e.g., a compound or a drug) that is required to produce infection, including a viral infection, in 50% of embryonated hen's eggs that are inoculated.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

Compounds described herein may comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., 3H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvate or water molecules can combine with the compounds according to the invention to form solvates and hydrates. Unless stated to the contrary, the invention includes all such possible solvates.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g., Almarasson, O., et al. (2004) The Royal Society of Chemistry, 1889-1896. Examples of co-crystals include p-toluenesulfonic acid and benzenesulfonic acid.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### 2. Pharmaceutical Compositions

Many proliferative disorders, such as tumors and cancers, have been shown to involve overexpression or upregulation of protein kinase activity. Protein kinases are kinase enzymes that modify proteins by chemically adding phosphate groups (phosphorylation). Phosphorylation usually results in a functional change of the target protein by changing enzyme activity, cellular location, or association with other proteins. Protein kinases can be subdivided or characterized by the amino acids of the target protein whose phosphorylation they control: most kinases act on both serine and threonine, the tyrosine kinases act on tyrosine, and a number (dual-specificity kinases) act on all three. There are also protein kinases that phosphorylate other amino acids, including histidine kinases that phosphorylate histidine residues. The human genome contains about 500 protein kinase genes and up to 30% of all human proteins may be modified by protein kinases. Kinases are known to regulate the majority of cellular pathways, especially those involved in signal transduction. Dysregulation of protein kinases by mutation, gene rearrangement, gene amplification, and overexpression of both receptor and ligand has been implicated in the development and progression of human cancers. Protein kinase inhibiting compounds or protein kinase inhibitors (PKIs) are therefore useful for treating diseases caused by or exacerbated by overexpression or upregulation of protein kinases. For example, tyrosine kinase inhibitors (TKIs also known as tyrphostins) have been shown to be effective anti-tumor agents and anti-leukemic agents (Lowery A et. al., Front Biosci. 2011 Jun. 1; 17:1996-2007).

Nilotinib is a tyrosine kinase inhibitor and was discovered by Novartis and is marketed as capsule dosage forms under the brand name Tasigna®. The solid form chosen for Tasigna® is Nilotinib hydrochloride (HCl) monohydrate. Nilotinib HCl can exist in a number of different solid forms including anhydrates, hydrates and solvates, all of which exhibit poor aqueous solubility.

It is known in the pharmaceutical arts that low-solubility drugs often show poor bioavailability or irregular absorption, the degree of irregularity being affected by factors such as dose level, fed state of the patient, and form of the drug.

In the low pH environment of the stomach (pH 1-2, usually about 1.2), Nilotinib is soluble. When the drug solution passes into the higher pH environment of the GI tract where the pH is 5 to 7, usually about 6.5, the drug may be above its equilibrium solubility at that pH. However, if the dose is relatively low and if the drug has the capacity to temporarily supersaturate, the drug may maintain supersaturation in the GI tract for a time, thus permitting absorption of the dissolved drug across the intestinal wall. In general, the residence time in the GI tract of humans is around 4 hours. Thus, a drug which can maintain supersaturation at intestinal pH will, in general, be better absorbed than one which does not.

Some basic drugs exhibit “dose/solubility-limited exposure.” As the dose is increased, the systemic drug exposure increases until a limiting dose is achieved, above which dose the increase in systemic exposure with increasing dose is less than that observed at doses lower than this dose. Since basic drugs are generally soluble at gastric pH, this effect may be due to precipitation of drug in the GI tract above the limiting dose.

Some drugs exhibit little or no capacity to be supersaturated at neutral pH; such drugs precipitate quickly in the GI tract even if reasonably soluble in the stomach, and are poorly bioavailable.

As is known in the art, it is not generally possible to predict the propensity of Nilotinib to supersaturate the small intestinal lumen.

Several approaches can be taken to improve aqueous solubility, including compositions aimed at retaining a basic drug in the supersaturated state upon transition from the acidic condition of the stomach to the neutral pH of the intestine. One of the most widely used approaches is salt formation of organic molecules with inorganic counter ions. The majority of such salts are crystalline and although they offer solubility improvement over their free acid or base counterparts, the solubility enhancement may not be sufficient for optimal bioavailability. In some cases more profound increase in solubility can be obtained by retaining the drug in a high energy amorphous form. The amorphous state is inherently unstable and hence there are numerous examples in the art of amorphous solid dispersions where polymeric excipients have been added to stabilize the amorphous state through the formation of hydrogen bonding and the like. However, hydrogen bonding as observed in various amorphous solid dispersions is not as strong as the ionic bonding often encountered in the crystalline salts, and as a result re-crystallization of drug molecule from an amorphous dispersion is a common problem. Hence, there is a need in the art for compositions which can more effectively stabilize an amorphous state in order to achieve the desired increase in solubility and bioavailability without compromising the physical stability of the drug.

In one aspect, the invention relates to a composition, such as a pharmaceutical composition, comprising a) Nilotinib, salified with b) a polymer selected from the group consisting of hydroxypropylmethyl-cellulose acetate succinate (HPMCAS), hydroxypropylmethylcellulose phthalate (HPMCP), cellulose acetate trimellitate (CAT), cellulose acetate phthalate (CAP), hydroxypropylcellulose acetate phthalate (HPCAP), hydroxypropylmethylcellulose acetate phthalate (HPMCAP), and methylcellulose acetate phthalate (MCAP); wherein the composition exhibits, in a dissolution experiment, at least a 1.25-fold improvement in solubility within the first 2 hours over a control composition comprising Nilotinib, not being salified with the polymer as measured according to a dissolution profile where FaSSIF is used as a medium and having a pH of 6.5.

Accordingly, the composition, such as the pharmaceutical composition, comprises a salt of Nilotinib and the polymer selected from the group consisting of hydroxypropylmethyl-cellulose acetate succinate (HPMCAS), hydroxypropylmethylcellulose phthalate (HPMCP), cellulose acetate trimellitate (CAT), cellulose acetate phthalate (CAP), hydroxypropylcellulose acetate phthalate (HPCAP), hydroxypropylmethylcellulose acetate phthalate (HPMCAP), and methylcellulose acetate phthalate (MCAP). Such a salt is a pharmaceutically acceptable salt of Nilotinib and the polymer selected from the group consisting of hydroxypropylmethyl-cellulose acetate succinate (HPMCAS), hydroxypropylmethylcellulose phthalate (HPMCP), cellulose acetate trimellitate (CAT), cellulose acetate phthalate (CAP), hydroxypropylcellulose acetate phthalate (HPCAP), hydroxypropylmethylcellulose acetate phthalate (HPMCAP), and methylcellulose acetate phthalate (MCAP). Accordingly, a pharmaceutically acceptable salt includes a salt of Nilotinib that retain the biological effectiveness and properties of Nilotinib.

Also disclosed herein is a solid dosage form comprising a salt of Nilotinib and a polymer wherein said solid dosage form releases at least 20% of the Nilotinib in 90 min in a dissolution study in FaSSIF in a dissolution bath at a pH of 6.5, utilizing a stirring rate of 100 rpm wherein the weight ratio of the amount of drug in said dosage form to the water is about 1/9. In this aspect, the polymer can be an acidic polymer. In this aspect, the acid polymer can be selected from the group consisting of hydroxypropylmethyl-cellulose acetate succinate (HPMCAS), hydroxypropylmethylcellulose phthalate (HPMCP), cellulose acetate trimellitate (CAT), cellulose acetate phthalate (CAP), hydroxypropylcellulose acetate phthalate (HPCAP), hydroxypropylmethylcellulose acetate phthalate (HPMCAP), methylcellulose acetate phthalate (MCAP). For example, the solid dosage form comprising a salt of Nilotinib and a polymer wherein said solid dosage form releases at least 25% of the Nilotinib in 90 min in a dissolution study in FaSSIF in a dissolution bath at a pH of 6.5, utilizing a stirring rate of 100 rpm wherein the weight ratio of the amount of drug in said dosage form to the water is about 1/9. In another example, the solid dosage form comprising a salt of Nilotinib and a polymer wherein said solid dosage form releases at least 30% of the Nilotinib in 90 min in a dissolution study in FaSSIF in a dissolution bath at a pH of 6.5, utilizing a stirring rate of 100 rpm wherein the weight ratio of the amount of drug in said dosage form to the water is about 1/9. The solid dosage form can comprise the aspects described for the composition disclosed herein.

Also disclosed herein is a composition of Nilotinib salified with HPMCP wherein an infrared spectrum of the composition does not have a N—H deformation peak at 1498 cm⁻¹ and do have a peak at about 1691 cm⁻¹.

In one aspect, the composition, such as the pharmaceutical composition, comprises an effective amount of Nilotinib. In a further aspect, the effective amount is a therapeutically effective amount. In a still further aspect, the effective amount is a prophylactically effective amount.

In one aspect, the composition comprises from about 1 mg to about 500 mg of Nilotinib. For example, the composition can comprise from about 1 mg to about 400 mg of Nilotinib. In another example, the composition can comprise from about 1 mg to about 300 mg of Nilotinib. In another example, the composition can comprise from about 1 mg to about 250 mg of Nilotinib. In another example, the composition can comprise from about 1 mg to about 200 mg of Nilotinib. In another example, the composition can comprise from about 1 mg to about 150 mg of Nilotinib. In another example, the composition can comprise from about 1 mg to about 100 mg of Nilotinib. In another example, the composition can comprise from about 1 mg to about 75 mg of Nilotinib. In another example, the composition can comprise from about 1 mg to about 50 mg of Nilotinib. In another example, the composition can comprise from about 1 mg to about 40 mg of Nilotinib. In another example, the composition can comprise from about 1 mg to about 30 mg of Nilotinib. In another example, the composition can comprise from about 1 mg to about 25 mg of Nilotinib. In another example, the composition can comprise from about 1 mg to about 20 mg of Nilotinib. In another example, the composition can comprise from about 1 mg to about 15 mg of Nilotinib. In another example, the composition can comprise from about 20 mg to about 200 mg of Nilotinib. In another example, the composition can comprise from about 20 mg to about 150 mg of Nilotinib. In another example, the composition can comprise from about 20 mg to about 100 mg of Nilotinib. In another example, the composition can comprise from about 1 mg to about 20 mg of Nilotinib. In another example, the composition can comprise from about 20 mg to about 50 mg of Nilotinib.

In one aspect, the polymer is hydroxypropylmethyl-cellulose acetate succinate (HPMCAS). In another aspect, the polymer is hydroxypropylmethylcellulose phthalate (HPMCP). In yet another aspect, the polymer is cellulose acetate trimellitate (CAT). In yet another aspect, the polymer is cellulose acetate phthalate (CAP). In another aspect, the polymer is hydroxypropylcellulose acetate phthalate (HPCAP). In another aspect, the polymer is hydroxypropylmethylcellulose acetate phthalate (HPMCAP). In another aspect, the polymer is methylcellulose acetate phthalate (MCAP). The aforementioned polymers, are commercially available in a variety of grades, which are all encompassed herein, For example, the polymers are available from Eastman Chemical: Co., Kingsport, Tenn.; and Shin Etsu, Tokyo, Japan. For example, HPMCAS is available from Shin Etsu in at least six different grades (LF, MF, HF, LG, MG, HG). It is contemplated that polymer can be of any suitable molecular weight, and have any suitable PDI.

In one aspect, the composition comprises from about 1 mg to about 10 g of the polymer. For example, the composition can comprise from about 25 mg to about 10 g of the polymer. In another example, the composition can comprise from about 50 mg to about 10 g of the polymer. In another example, the composition can comprise from about 100 mg to about 10 g of the polymer. In another example, the composition can comprise from about 500 mg to about 10 g of the polymer. In another example, the composition can comprise from about 1 g to about 10 g of the polymer. In another example, the composition can comprise from about 3 g to about 10 g of the polymer. In another example, the composition can comprise from about 5 g to about 10 g of the polymer. In another example, the composition can comprise from about 1 mg to about 5 g of the polymer. In another example, the composition can comprise from about 1 mg to about 3 g of the polymer. In another example, the composition can comprise from about 1 mg to about 1 g of the polymer. In another example, the composition can comprise from about 1 mg to about 500 mg of the polymer.

In one aspect, the composition comprises a weight ratio of Nilotinib:polymer from 20:1 to 1:20. For example, the composition can comprises a weight ratio of Nilotinib:polymer from 15:1 to 1:20. In another example, the composition can comprises a weight ratio of Nilotinib:polymer from 10:1 to 1:20. In another example, the composition can comprises a weight ratio of Nilotinib:polymer from 5:1 to 1:20. In another example, the composition can comprise a weight ratio of Nilotinib:polymer from 1:1 to 1:20. In another example, the composition can comprise a weight ratio of Nilotinib:polymer from 20:1 to 1:15. In another example, the composition can comprise a weight ratio of Nilotinib:polymer from 20:1 to 1:10. In another example, the composition can comprise a weight ratio of Nilotinib:polymer from 20:1 to 1:5. In another example, the composition can comprise a weight ratio of Nilotinib:polymer from 20:1 to 1:1. In another example, the composition can comprise a weight ratio of Nilotinib:polymer from 10:1 to 1:10. In another example, the composition can comprise a weight ratio of Nilotinib:polymer from 5:1 to 1:5. In another example, the composition can comprise a weight ratio of Nilotinib:polymer from 2:1 to 1:2.

In one aspect, the composition has an average particle size range from greater than about 1 μm to about 1,000 μm. For example the composition can have an average particle size range from about 1.5 μm to about 1,000 μm. In another example, the composition does not have an average particle size of less than 1,000 nm. In another example, the composition does not have an average particle size of less than 1.5 μm. In another example, the composition can have an average particle size range from greater than about 1 μm to about 500 μm. In another example, the composition can have an average particle size range from greater than about 1 μm to about 300 μm. In another example, the composition can have an average particle size range from greater than about 1 μm to about 100 μm. In another example, the composition can have an average particle size range from greater than about 1 μm to about 75 μm. In another example, the composition can have an average particle size range from greater than about 1 μm to about 50 μm. In another example, the composition can have an average particle size range from greater than about 1 μm to about 30 μm. In another example, the composition can have an average particle size range from greater than about 1 μm to about 20 μm. In another example, the composition can have an average particle size range from greater than about 1 μm to about 10 μm.

In one aspect, the control composition is Nilotinib, salified with HCl. In another aspect, the control composition is a freebase of Nilotinib. In another aspect, the control composition is Nilotinib, salified with hydrochloride, fumarate, 2-chloromandelate, succinate, adipate, L-tartrate, glutarate, p-toluenesulfonate, camphorsulfonate, glutamate, palmitate, quinate, citrate, maleate, acetate, L-malate, L-aspartate, formate, hydrobromide, oxalate, malonatec, benzenesulfonic acid, butanedisulfonic acid, 1-5-naphthalenedisulfonic acid, naphthalene-1-sulfonic acid, or 1-hydroxynaph-thoic acid. For example, the control composition is Nilotinib, salified with hydrochloride. In another example, the control composition is Nilotinib, salified with fumarate. In another example, the control composition is Nilotinib, salified with 2-chloromandelate. In another example, the control composition is Nilotinib, salified with succinate. In another example, the control composition is Nilotinib, salified with adipate. In another example, the control composition is Nilotinib, salified with L-tartrate. In another example, the control composition is Nilotinib, salified with glutarate. In another example, the control composition is Nilotinib, salified with p-toluenesulfonate. In another example, the control composition is Nilotinib, salified with camphorsulfonate. In another example, the control composition is Nilotinib, salified with glutamate. In another example, the control composition is Nilotinib, salified with palmitate. In another example, the control composition is Nilotinib, salified with quinate. In another example, the control composition is Nilotinib, salified with citrate. In another example, the control composition is Nilotinib, salified with maleate. In another example, the control composition is Nilotinib, salified with acetate. In another example, the control composition is Nilotinib, salified with L-malate. In another example, the control composition is Nilotinib, salified with L-aspartate. In another example, the control composition is Nilotinib, salified with formate. In another example, the control composition is Nilotinib, salified with hydrobromide. In another example, the control composition is Nilotinib, salified with oxalate. In another example, the control composition is Nilotinib, salified with malonatec. In another example, the control composition is Nilotinib, salified with benzenesulfonic acid. In another example, the control composition is Nilotinib, salified with butanedisulfonic acid. In another example, the control composition is Nilotinib, salified with 1-5-naphthalenedisulfonic acid. In another example, the control composition is Nilotinib, salified with naphthalene-1-sulfonic acid. In another example, the control composition is Nilotinib, salified with 1-hydroxynaph-thoic acid.

In one aspect, the composition is an amorphous dispersion. In one aspect, the composition is non-crystalline.

In one aspect, the composition exhibits, in a dissolution experiment, at least a 1.5-fold improvement in solubility within the first 2 hours over a control composition comprising Nilotinib, not being salified with the polymer as measured according to a dissolution profile where the medium is FaSSIF having a pH of 6.5. In another aspect, the composition exhibits, in a dissolution experiment, at least a 2.0-fold improvement in solubility within the first 2 hours over a control composition comprising Nilotinib, not being salified with the polymer as measured according to a dissolution profile where the medium is FaSSIF having a pH of 6.5. In another aspect, the composition exhibits, in a dissolution experiment, at least a 3.0-fold improvement in solubility within the first 2 hours over a control composition comprising Nilotinib, not being salified with the polymer as measured according to a dissolution profile where the medium is FaSSIF having a pH of 6.5. In another aspect, the composition exhibits, in a dissolution experiment, from a 1.25 to a 3.0-fold improvement in solubility within the first 2 hours over a control composition comprising Nilotinib, not being salified with the polymer as measured according to a dissolution profile where the medium is FASSIF having a pH of 6.5.

In one aspect, the disclosed composition can be tested in vivo and wherein the Cmax achieved with said composition is at least 1.25-fold, preferably at least 1.5-fold, more preferably at least 2-fold the Cmax achieved with a control composition comprising Nilotinib, not being salified with the polymer. Cmax is well understood in the art as an abbreviation for the maximum drug concentration in serum or plasma of the test subject. In vivo testing protocols can be designed in a number of ways. By measuring the Cmax for a population to which the test composition has been administered and comparing it with the Cmax for the same population to which the control has also been administered, the test composition can be evaluated.

In one aspect, the disclosed composition can exhibit at least a 1.25-fold, preferably at least a 1.5-fold, more preferably at least a 2-fold improvement in AUC over a control not containing polymer. AUC is a determination of the area under the curve (AUC) plotting the serum or plasma concentration of drug along the ordinate (Y-axis) against time along the abscissa (X-axis). Generally, the values for AUC represent a number of values taken from all the subjects in a patient test population and are, therefore, mean values averaged over the entire test population. By measuring the AUC for a population to which the test composition has been administered and comparing it with the AUC for the same population to which the control has been administered, the test composition can be evaluated. Alternatively, the AUC test/AUC control ratio may be determined for each subject, then averaged. AUC's are well understood, frequently used tools in the pharmaceutical arts and have been extensively described, for example in “Pharmacokinetics Processes and Mathematics”, Peter E. Welling, ACS Monograph 185; 1986.”

Thus, a disclosed composition effects either a Cmax and/or an AUC that is at least 1.25 times, preferably at least 1.5 times, more preferably at least 2.0 times the corresponding Cmax or AUC exhibited by a control composition comprising Nilotinib, not being salified with the polymer. For example, the disclosed composition displays at least a 1.25-fold improvement in Cmax as discussed above, and also exhibit at least a 1.25-fold improvement in AUC.

Cmax and AUC can be determined in humans or a suitable animal model, such as dogs or rats.

The composition disclosed herein can be tested in vitro to determine if it exhibits an equilibrium solubility in a pH 1.0 to 2.0 use environment that is at least 3-fold its equilibrium solubility in a pH 5.0 to 7.0 environment. The composition disclosed herein can be dissolved in a pH 1-2 environment, typically aqueous deionized distilled water adjusted to a target pH within the aforesaid pH 1-2 range by adding an appropriate amount of hydrochloric acid. The amount of composition added is an amount sufficient to saturate the aqueous test medium. The test medium can be agitated, typically gently, by means of a stirring bar, overhead stirrer, or the like. Typically the test medium is left to sit (while being agitated) for several hours, typically overnight. The sample can then be filtered or centrifuged as previously described, and solubility in the filtrate or supernatant can then be measured by determining the concentration with any suitable means of detection appropriate to the drug. This entire experiment can be done in a dissolution bath or the like. Likewise, the solubility is also determined at pH 5 to 7. In one aspect, if the composition can have a pH 1-2 solubility that is 3- or more-fold to its pH 5-7 solubility. For example, a quantity of Nilotinib powder as base or salt or either equivalent of 150 mg base can be dissolved in the test medium, an aqueous use environment having a pH of 1.0-2.0, usually 500-900 mL. Generally a single pH within the range, for example a pH of 1.0, is chosen for consistency in results and to facilitate comparison. Alternatively a quantity of Nilotinib powder as base or salt or either equivalent of 40 mg base is filled into a size 1 gelatin capsule, which is then dissolved in the test medium, an aqueous use environment having a pH of 1.0-2.0, usually in a volume of 500-900 mL. Generally a single pH within the range, for example a pH of 1.0, is chosen for consistency in results and to facilitate comparison.

The disclosed composition can also be tested in vitro. A typical test can be described as follows for a planned dosage form. A quantity of Nilotinib of either, usually on the order of 1-5 mg, is dissolved in, as the test medium, an aqueous use environment having a pH of 1.0-2.0, usually 5-40 mL. Generally a single pH within the range, for example a pH of 1.2, is chosen for consistency in results and to facilitate comparison. The composition may or may not dissolve completely. The aqueous environment is, as disclosed above, typically deionized, distilled water with sufficient aqueous hydrochloric acid added to adjust the pH to 1.0-2.0. Acid having a normality of 1 to 4 is usually sufficient for adjusting the pH to within 1.0 to 2.0, although a higher concentration can be used if desired. Sufficient acid is present in the test medium such that at least a portion of the drug dissolves while still maintaining the pH of the test medium within a range of 1.0-2.0. It is desirable to agitate the test medium, as by using a stirring bar or an overhead stirrer, and the medium is allowed to stir up to several hours or longer, if desired. An identical control sample can be made in the same manner or, alternatively, the drug containing sample test medium already made can, before the addition of any polymer, be split into two equal portions, one being reserved as the control, the other as the test sample. At this point a quantity of test polymer should be added to the test sample in proportion to its intended presence in the final composition. Polymer is omitted from the control sample, although other (non-polymer) excipients can be added.

Control and test can then be evaluated in a standard pH between 5.0 and 7.0, a standard target pH usually being chosen, for example pH 6.5. Generally a single pH within the range is chosen for consistency in results and to facilitate comparison. The composition may or may not dissolve completely. The aqueous environment is, as disclosed above, typically deionized, distilled water buffered with a suitable buffering agent for example potassium dihydrogen phosphate adjusted with a sufficient amount of aqueous sodium (or potassium) hydroxide to achieve a pH of 5.0-7.0. Sufficient buffering agent is present in the test medium such that at least a portion of the drug dissolves while still maintaining the pH of the test medium within a range of 5.0-7.0. It is desirable to agitate the test medium, as by using a stirring bar or an overhead stirrer, and the medium is allowed to stir up to several hours or longer, if desired. The sample and control can then be filtered (or centrifuged) and the filtrate (or supernate) analyzed by any convenient technique suitable to the drug being tested, such as HPLC, GC, and so forth, using appropriate detection. In one aspect, the concentration detected at pH 5.0 to 7.0 in the presence of polymer is at least 1.5 times the concentration of that in the control at any time during the 2 hours following titration to pH 5 to 7.

The test described above can also be conducted for a pre-formed or pre-manufactured dosage (e.g., a tablet or capsule) already containing polymer. The test is as described above, with a few modifications. First, it may be necessary to pulverize the dosage form if it is a tablet. If the dosage form is a capsule or a powder for oral suspension, then the capsule fill or powder may be tested directly. Since a pre-formed test sample contains test polymer, it will not be possible to split the initial sample into a test portion and control portion. It may accordingly be necessary to make a like composition less the polymer to function as a control. Alternatively, if none of the excipients influences solubility, the control can consist of drug alone, i.e., no other excipients. Generally, initial identical aqueous test medium solutions having a pH of 1.0 to 2.0 should be made, or divided as aliquots out of a common stock, and set aside. Identical quantities of test and control compositions can be added to each and then treated in parallel, as described above.

The composition can further comprise a pharmaceutically acceptable carrier. A pharmaceutically acceptable carrier refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. The compounds can be formulated with pharmaceutically acceptable carriers or diluents as well as any other known adjuvants and excipients in accordance with conventional techniques such as those disclosed in Remington: The Science and Practice of Pharmacy, 19th Edition, Gennaro, Ed., Mack Publishing Co., Easton, Pa., 1995.

In various aspects, the disclosed compositions comprise the salt of Nilotinib as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

The compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

In various aspects, the pharmaceutical compositions disclosed herein can further include one or more other therapeutically active compounds in combination with the salt of Nilotinib and the polymer selected from the group consisting of hydroxypropylmethyl-cellulose acetate succinate (HPMCAS), hydroxypropylmethylcellulose phthalate (HPMCP), cellulose acetate trimellitate (CAT), cellulose acetate phthalate (CAP), hydroxypropylcellulose acetate phthalate (HPCAP), hydroxypropylmethylcellulose acetate phthalate (HPMCAP), and methylcellulose acetate phthalate (MCAP).

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, and/or pharmaceutically acceptable salts thereof, can also be prepared in powder or liquid concentrate form.

In one aspect, the composition further comprises one or more excipients selected from the group consisting of colloidal silica, a lubricant, a filler, a disintegrant, a plasticizer, a coloring agent, an emulsifying agent, a diluent, a flavoring agent, a binder, a film forming polymer, an antioxidant, a light stabilizer, a radical scavenger, a surface active agent, a pH modifier, a drug complexing agent, and a stabilizer against microbial attack, or a combination thereof.

As described herein, compositions of this invention can be used in a wide variety of forms for administration of drugs orally, usually together with a pharmaceutically acceptable diluent or carrier. Exemplary dosage forms are powders or granules that can be taken orally either dry or reconstituted by addition of water to form a paste, slurry, suspension or solution; tablets, capsules, or pills. Various additives can be mixed, or granulated with the compositions of this invention to form a material suitable for the above dosage forms. Potentially beneficial additives fall generally into the following classes: other matrix materials or diluents, surface active agents, drug complexing agents or solubilizers, fillers, disintegrants, binders, lubricants, and pH modifiers (e.g., acids, bases, or buffers).

Non-limiting examples of other matrix materials, fillers, or diluents include lactose, mannitol, xylitol, microcrystalline cellulose, calcium diphosphate, and starch.

Non-limiting examples of surface active agents include sodium lauryl sulfate and polysorbate 80. The surface-active agent can be a fatty acid and alkyl sulfonate; commercial surfactants such as those sold under tradenames such as benzethanium chloride (Hyamine® 1622, available from Lonza, Inc., Fairlawn, N.J.), docusate sodium (available from Mallinckrodt Spec. Chem., St. Louis, Mo.), polyoxyethylene sorbitan fatty acid esters (Tween®, available from ICI Americas Inc, Wilmington, Del.), Liposorb® P-20 (available from Lipochem Inc, Patterson, N.J.), Capmul® POE-0 (available from Abitec Corp., Janesville, Wis.), and natural surfactants such as sodium taurocholic acid, 1-palmitoyl-2-oleoyl-sn-glycero-3-phosphocholine, lecithin, and other phospholipids and mono- and diglycerides. Such materials can be employed advantageously to increase the rate of dissolution by facilitating wetting, thereby increasing the maximum drug concentration and the degree of supersaturation attained, and also to inhibit crystallization or precipitation of drug by interacting with dissolved drug by mechanisms such as complexation, formation of inclusion complexes, formation of micelles or adsorbing to the surface of solid drug, crystalline or amorphous. These surface active agents can comprise up to 25% of the disclosed composition.

Non-limiting examples of drug complexing agents or solubilizers include the polyethylene glycols, caffeine, xanthene, gentisic acid and cylodextrins.

Non-limiting examples of disintegrants include sodium starch gycolate, sodium alginate, carboxymethyl cellulose sodium, methyl cellulose, and croscarmellose sodium.

Non-limiting examples of binders include methyl cellulose, microcrystalline cellulose, starch, and gums such as guar gum, and tragacanth.

Non-limiting examples of lubricants include magnesium stearate and calcium stearate.

Non-limiting examples of pH modifiers include acids such as citric acid, acetic acid, ascorbic acid, lactic acid, aspartic acid, succinic acid, phosphoric acid, and the like; and buffers generally comprising mixtures of acids and the salts of said acids.

As previously mentioned, for oral administration a pharmaceutical composition suitable for use in this invention can take various forms, including solutions, suspensions, tablets, pills, capsules, powders, and the like. Tablets may contain various excipients such as the matrix materials, fillers, diluents, surface active agents, drug complexing agents, solubilizers, disintegrants, binders, lubricants, and pH modifiers exemplified above. Hard gelatin capsule formulations generally comprise drug, polymer, and excipients as described above for tablets. When aqueous suspensions and/or elixirs are desired for oral administration, the compounds of this invention can be combined with various sweetening agents, flavoring agents, coloring agents, emulsifying agents and/or suspending agents, as well as such diluents as water, ethanol, propylene glycol, glycerin and various like combinations thereof.

In a further aspect, the composition is formulated for parenteral administration. In a still further aspect, the composition is formulated for inhalation. In yet a further aspect, the composition is formulated for oral administration. In an even further aspect, the composition is formulated for topical administration.

The disclosed composition and solid dosage forms have faster dissolution profiles and higher solubility, are expected to have higher bioavailability, and reduced food effects compared a control composition comprising Nilotinib, not being salified with the polymer.

The disclosed composition and solid dosage forms can have excellent stability and, in particular, exhibit high resistance against recrystallization or decomposition of the Nilotinib. Thus, upon storage for 6 weeks at 40° C. and 75% humidity (e.g., when kept in high density polyethylene (HDPE) bottles without desiccant). In one aspect, the disclosed composition and solid dosage forms do not exhibit any sign of crystallinity (as evidenced by DSC or WAXS analysis) and will contain at least about 98% of the initial Nilotinib content (which can be evidenced by HPLC analysis).

### 3. Methods

In one aspect, also disclosed herein are method of using the compositions, such as the pharmaceutical compositions, disclosed herein. Disclosed is a method of treating a disorder of uncontrolled cellular proliferation, the method comprising the step of administering to the subject an effective amount of a composition disclosed herein.

In one aspect, the disorder of uncontrolled cellular proliferation is selected from tumors and cancers including, but not limited to, neurofibromatosis, tuberous sclerosis, hemangiomas and lymphangiogenesis, cervical, anal and oral cancers, eye or ocular cancer, stomach cancer, colon cancer, bladder cancer, rectal cancer, liver cancer, pancreas cancer, lung cancer, breast cancer, cervix uteri cancer, corpus uteri cancer, ovary cancer, prostate cancer, testis cancer, renal cancer, brain cancer, cancer of the central nervous system, head and neck cancer, throat cancer, skin melanoma, acute lymphocytic leukemia, acute myelogenous leukemia, Ewing's Sarcoma, Kaposi's Sarcoma, basal cell carcinoma and squamous cell carcinoma, small cell lung cancer, choriocarcinoma, rhabdomyosarcoma, angiosarcoma, hemangioendothelioma, Wilms Tumor, neuroblastoma, mouth/pharynx cancer, esophageal cancer, larynx cancer, lymphoma, multiple myeloma; cardiac hypertrophy, age-related macular degeneration and diabetic retinopathy. For example, the disorder of uncontrolled cellular proliferation can be leukemia. In another example, the disorder of uncontrolled cellular proliferation can be chronic myelogenous leukemia.

To treat or control the disorder of uncontrolled cellular proliferation, the compositions and pharmaceutical compositions disclosed herein are administered to a subject in need thereof, such as a vertebrate, e.g., a mammal, a fish, a bird, a reptile, or an amphibian. The subject can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. The subject is preferably a mammal, such as a human. Prior to administering the compounds or compositions, the subject can be diagnosed with a need for treatment of a viral infection, such as influenza virus. In one aspect, the subject has been diagnosed with the disorder of uncontrolled cellular proliferation. For example, the subject can have been diagnosed with a need for treatment of a disorder of uncontrolled cellular proliferation prior to the administering step.

The compositions disclosed herein can be administered to the subject according to any method. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration and parenteral administration, including injectable such as intravenous administration, intra-arterial administration, intramuscular administration, and subcutaneous administration. Administration can be continuous or intermittent. A preparation can be administered therapeutically; that is, administered to treat an existing disease or condition. A preparation can also be administered prophylactically; that is, administered for prevention of an infection or condition, such as influenza virus. For example, the administration can be oral. For oral administration the compositions can be presented as discrete units such as capsules, cachets or tablets (pills), each containing a predetermined amount of Nilotinib. As disclosed herein, tablets (pills) and capsules for oral administration may contain conventional excipients such as binding agents, fillers, lubricants, disintegrants, or wetting agents. The tablets (pills) can be coated according to methods well known in the art.

The therapeutically effective amount or dosage of the compound can vary within wide limits. Such a dosage is adjusted to the individual requirements in each particular case including the specific compound(s) being administered, the route of administration, the condition being treated, as well as the patient being treated. In general, in the case of oral or parenteral administration to adult humans weighing approximately 70 Kg or more, a daily dosage of about 10 mg to about 10,000 mg, preferably from about 200 mg to about 1,000 mg, should be appropriate, although the upper limit may be exceeded. The daily dosage can be administered as a single dose or in divided doses, or for parenteral administration, as a continuous infusion. Single dose compositions can contain such amounts or submultiples thereof of the compound or composition to make up the daily dose. The dosage can be adjusted by the individual physician in the event of any contraindications. Dosage can vary, and can be administered in one or more dose administrations daily, for one or several days.

In one aspect, the daily dosage can be amount Nilotinib present in the disclosed compositions. For example, the daily dosage can comprises from about 1 mg to about 500 mg of Nilotinib. For example, the daily dosage can comprise from about 1 mg to about 400 mg of Nilotinib. In another example, the daily dosage can comprise from about 1 mg to about 300 mg of Nilotinib. In another example, the daily dosage can comprise from about 1 mg to about 250 mg of Nilotinib. In another example, the daily dosage can comprise from about 1 mg to about 200 mg of Nilotinib. In another example, the daily dosage can comprise from about 1 mg to about 150 mg of Nilotinib. In another example, the daily dosage can comprise from about 1 mg to about 100 mg of Nilotinib. In another example, the daily dosage can comprise from about 1 mg to about 75 mg of Nilotinib. In another example, the daily dosage can comprise from about 1 mg to about 50 mg of Nilotinib. In another example, the daily dosage can comprise from about 1 mg to about 40 mg of Nilotinib. In another example, the daily dosage can comprise from about 1 mg to about 30 mg of Nilotinib. In another example, the daily dosage can comprise from about 1 mg to about 25 mg of Nilotinib. In another example, the daily dosage can comprise from about 1 mg to about 20 mg of Nilotinib. In another example, the daily dosage can comprise from about 1 mg to about 15 mg of Nilotinib. In another example, the daily dosage can comprise from about 20 mg to about 200 mg of Nilotinib. In another example, the daily dosage can comprise from about 20 mg to about 150 mg of Nilotinib. In another example, the daily dosage can comprise from about 20 mg to about 100 mg of Nilotinib. In another example, the daily dosage can comprise from about 1 mg to about 20 mg of Nilotinib. In another example, the daily dosage can comprise from about 20 mg to about 50 mg of Nilotinib.

In a further aspect, the effective amount is a therapeutically effective amount. In a still further aspect, the effective amount is a prophylactically effective amount.

In one aspect, the disclosed method further comprises the step of identifying a subject in need of treatment of a disorder of uncontrolled cellular proliferation.

### 4. Kits

In one aspect, the invention relates to a kit comprising an effective amount of a composition disclosed herein, and one or more of: (a) another tyrosine kinase inhibitor; (b) means for administering the composition; (c) instructions for use; (d) a drug for treatment of a disorder of uncontrolled cellular proliferation. The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

In one aspect, the disorder of uncontrolled cellular proliferation is selected from tumors and cancers including, but not limited to, neurofibromatosis, tuberous sclerosis, hemangiomas and lymphangiogenesis, cervical, anal and oral cancers, eye or ocular cancer, stomach cancer, colon cancer, bladder cancer, rectal cancer, liver cancer, pancreas cancer, lung cancer, breast cancer, cervix uteri cancer, corpus uteri cancer, ovary cancer, prostate cancer, testis cancer, renal cancer, brain cancer, cancer of the central nervous system, head and neck cancer, throat cancer, skin melanoma, acute lymphocytic leukemia, acute myelogenous leukemia, Ewing's Sarcoma, Kaposi's Sarcoma, basal cell carcinoma and squamous cell carcinoma, small cell lung cancer, choriocarcinoma, rhabdomyosarcoma, angiosarcoma, hemangioendothelioma, Wilms Tumor, neuroblastoma, mouth/pharynx cancer, esophageal cancer, larynx cancer, lymphoma, multiple myeloma; cardiac hypertrophy, age-related macular degeneration and diabetic retinopathy. For example, the disorder of uncontrolled cellular proliferation can be leukemia. In another example, the disorder of uncontrolled cellular proliferation can be chronic myelogenous leukemia.

In a further aspect, the at least one compound and the at least one agent are co-packaged. In a still further aspect, the at least one compound and the at least one agent are co-formulated.

In a further aspect, the kit further comprises a plurality of dosage forms, the plurality comprising one or more doses; wherein each dose comprises an effective amount of Nilotinib. In a still further aspect, the effective amount is a therapeutically effective amount. In yet a further aspect, the effective amount is a prophylactically effective amount.

In a further aspect, the dosage forms are formulated for oral administration, inhalation, topical administration, and/or parenteral administration. In a still further aspect, the dosage form for the at least one compound is formulated for oral administration and the dosage form for the at least one agent is formulated for parental administration. In yet a further aspect, the dosage form for the composition is formulated for parental administration and the dosage form for the at least one agent is formulated for oral administration. In an even further aspect, the dosage form for the composition is formulated for topical administration and the dosage form for the composition is formulated for parental administration. In a still further aspect, the dosage form for the composition is formulated for parental administration and the dosage form for the composition is formulated for topical administration. In yet a further aspect, the dosage form for the composition is formulated for oral administration and the dosage form for the composition is formulated for inhalation. In an even further aspect, the dosage form for the composition is formulated for inhalation and the dosage form for the composition is formulated for oral administration. In a still further aspect, the dosage form for the composition is formulated for topical administration and the dosage form for the composition is formulated for inhalation. In a yet further aspect, the dosage form for the composition is formulated for inhalation and the dosage form for the composition is formulated for topical administration.

It is understood that the disclosed kits can be prepared from the disclosed compounds, products, and pharmaceutical compositions. It is also understood that the disclosed kits can be employed in connection with the disclosed methods of using.

### 5. Aspects

In view of the described systems and methods and variations thereof, herein below are described certain more particularly described aspects of the inventions. These particularly recited aspects should not however be interpreted to have any limiting effect on any different claims containing different or more general teachings described herein, or that the “particular” aspects are somehow limited in some way other than the inherent meanings of the language and formulas literally used therein.
Aspect 1: A composition comprising: a) Nilotinib, salified with b) a polymer selected from the group consisting of hydroxypropylmethyl-cellulose acetate succinate (HPMCAS), hydroxypropylmethylcellulose phthalate (HPMCP), cellulose acetate trimellitate (CAT), cellulose acetate phthalate (CAP), hydroxypropylcellulose acetate phthalate (HPCAP), hydroxypropylmethylcellulose acetate phthalate (HPMCAP), and methylcellulose acetate phthalate (MCAP); wherein the composition exhibits, in a dissolution experiment, at least a 1.25-fold improvement in solubility within the first 2 hours over a control composition comprising Nilotinib, not being salified with the polymer as measured according to a dissolution profile where FaSSIF is used as a medium and having a pH of 6.5.
Aspect 2: The composition of aspect 1, wherein the Nilotinib is molecularly dispersed in the polymer.
Aspect 3: The composition of any one of aspects 1-2, wherein the polymer is HPMCP or HPMCAS.
Aspect 4: The composition of any one of aspects 1-3, wherein the polymer is HPMCP.
Aspect 5: The composition of any one of aspects 1-4, wherein the composition exhibits, in a dissolution experiment, at least a 1.5-fold improvement in solubility within the first 2 hours over a control composition comprising Nilotinib, not being salified with the polymer as measured according to a dissolution profile where the medium is FaSSIF having a pH of 6.5.
Aspect 6: The composition of any one of aspects 1-5, wherein the composition exhibits, in a dissolution experiment, at least a 2.0-fold improvement in solubility within the first 2 hours over a control composition comprising Nilotinib, not being salified with the polymer as measured according to a dissolution profile where the medium is FaSSIF having a pH of 6.5.
Aspect 7: The composition of any one of aspects 1-6, wherein the composition has an average particle size range from greater than about 1 μm to about 1,000 μm.
Aspect 8: The composition of any one of aspects 1-7, wherein the composition has an average particle size range from 1 μm to 20 μm.
Aspect 9: The composition of any one of aspects 1-8, wherein the composition does not have an average particle size of less than 1,000 nm.
Aspect 10: The composition of any one of aspects 1-9, wherein the control composition is Nilotinib, salified with HCl.
Aspect 11: The composition of any one of aspects 1-10, wherein the control composition is a freebase of Nilotinib.
Aspect 12: The composition of any one of aspects 1-11, wherein the control composition is Nilotinib, salified with hydrochloride, fumarate, 2-chloromandelate, succinate, adipate, L-tartrate, glutarate, p-toluenesulfonate, camphorsulfonate, glutamate, palmitate, quinate, citrate, maleate, acetate, L-malate, L-aspartate, formate, hydrobromide, oxalate, malonatec, benzenesulfonic acid, butanedisulfonic acid, 1-5-naphthalenedisulfonic acid, naphthalene-1-sulfonic acid, or 1-hydroxynaph-thoic acid.
Aspect 13: The composition of any one of aspects 1-12, wherein the composition further comprises a pharmaceutically acceptable carrier or diluent.
Aspect 14: The composition of any one of aspects 1-13, wherein the composition further comprises a pharmaceutically acceptable carrier.
Aspect 15: The composition of any one of aspects 1-14, wherein the composition is in oral dosage form.
Aspect 16: The composition of any one of aspects 1-15, wherein the composition comprises from about 1 mg to about 10 g of the polymer.
Aspect 17: The composition of any one of aspects 1-16, wherein the composition comprises from about 20 mg to about 1 g of the polymer.
Aspect 18: The composition of any one of aspects 1-17, wherein the composition further comprises one or more excipients.
Aspect 19: The composition of any one of aspects 1-18, wherein the composition further comprises one or more excipients selected from the group consisting of colloidal silica, a lubricant, a filler, a disintegrant, a plasticizer, a coloring agent, an emulsifying agent, a diluent, a flavoring agent, a binder, a film forming polymer, an antioxidant, a light stabilizer, a radical scavenger, a surface active agent, a pH modifier, a drug complexing agent, and a stabilizer against microbial attack, or a combination thereof.
Aspect 20: The composition of any one of aspects 1-19, wherein the composition comprises from about 1 mg to about 500 mg of Nilotinib.
Aspect 21: The composition of any one of aspects 1-20, wherein the composition comprises from about 20 mg to about 200 mg of Nilotinib.
Aspect 22: The composition of any one of aspects 1-21, wherein the composition comprises from about 1 mg to about 50 mg of Nilotinib.
Aspect 23: The composition of any one of aspects 1-22, wherein the composition comprises from about 1 mg to about 40 mg of Nilotinib.
Aspect 24: The composition of any one of aspects 1-23, wherein the composition comprises from about 1 mg to about 30 mg of Nilotinib.
Aspect 25: The composition of any one of aspects 1-24, wherein the composition comprises a weight ratio of Nilotinib:polymer from 20:1 to 1:20.
Aspect 26: The composition of any one of aspects 1-25, wherein the composition comprises a weight ratio of Nilotinib:polymer from 10:1 to 1:10.
Aspect 27: The composition of any one of aspects 1-26, wherein the composition is an amorphous dispersion.
Aspect 28: A composition comprising a salt of Nilotinib and a polymer wherein said composition releases at least 20% of the Nilotinib in 90 min in a dissolution study in FaSSIF in a dissolution bath at a pH of 6.5, utilizing a stirring rate of 100 rpm wherein the weight ratio of the amount of Nilotinib in said dosage form to the water is about 1/9.
Aspect 29: A composition of Nilotinib salified with HPMCP wherein an infrared spectrum of the composition does not have a N—H deformation peak at 1498 cm⁻¹ and do have a peak at about 1691 cm⁻¹.
Aspect 30: A method for treating a disorder of uncontrolled cellular proliferation, the method comprising the step of administering to the subject an effective amount of a composition comprising: a) Nilotinib, salified with b) a polymer selected from the group consisting of hydroxypropylmethyl-cellulose acetate succinate (HPMCAS), hydroxypropylmethylcellulose phthalate (HPMCP), cellulose acetate trimellitate (CAT), cellulose acetate phthalate (CAP), hydroxypropylcellulose acetate phthalate (HPCAP), hydroxypropylmethylcellulose acetate phthalate (HPMCAP), and methylcellulose acetate phthalate (MCAP).
Aspect 31: The method of aspect 30, wherein the disorder of uncontrolled cellular proliferation is a leukemia.
Aspect 32: The method of any one of aspects 30-31, wherein the subject has been diagnosed with the disorder of uncontrolled cellular proliferation.
Aspect 33: The method of any one of aspects 30-32, wherein the disorder of uncontrolled cellular proliferation is selected from neurofibromatosis, tuberous sclerosis, hemangiomas and lymphangiogenesis, cervical, anal cancer, oral cancer, eye or ocular cancer, stomach cancer, colon cancer, bladder cancer, rectal cancer, liver cancer, pancreas cancer, lung cancer, breast cancer, cervix uteri cancer, corpus uteri cancer, ovary cancer, prostate cancer, testis cancer, renal cancer, brain cancer, cancer of the central nervous system, head and neck cancer, throat cancer, skin melanoma, acute lymphocytic leukemia, acute myelogenous leukemia, Ewing's Sarcoma, Kaposi's Sarcoma, basal cell carcinoma and squamous cell carcinoma, small cell lung cancer, choriocarcinoma, rhabdomyosarcoma, angiosarcoma, hemangioendothelioma, Wilms Tumor, neuroblastoma, mouth/pharynx cancer, esophageal cancer, larynx cancer, lymphoma, multiple myeloma; cardiac hypertrophy, age-related macular degeneration and diabetic retinopathy.
Aspect 34: The method of any one of aspects 30-33, wherein the cancer is chronic myelogenous leukemia.
Aspect 35: The method of any one of aspects 30-34, wherein the effective amount is a therapeutically effective amount.
Aspect 36: The method of any one of aspects 30-35, wherein the effective amount is a prophylactically effective amount.
Aspect 37: The method of any one of aspects 30-36, wherein the subject is a human.
Aspect 38: The method of any one of aspects 30-37, wherein the method further comprises the step of identifying a subject in need of treatment of a disorder of uncontrolled cellular proliferation.
Aspect 39: The method of any one of aspects 30-38, the mammal has been diagnosed with a need for treatment of a disorder of uncontrolled cellular proliferation prior to the administering step.
Aspect 40: The method of any one of aspects 30-39, wherein the Nilotinib is molecularly dispersed in the polymer.
Aspect 41: The method of any one of aspects 30-40, wherein the polymer is HPMCP or HPMCAS.
Aspect 42: The method of any one of aspects 30-41, wherein the polymer is HPMCP.
Aspect 43: The method of any one of aspects 30-42, wherein the composition exhibits, in a dissolution experiment, at least a 1.5-fold improvement in solubility within the first 2 hours over a control composition comprising Nilotinib, not being salified with the polymer as measured according to a dissolution profile where the medium is FASSIF having a pH of 6.5.
Aspect 44: The method of any one of aspects 30-43, wherein the composition exhibits, in a dissolution experiment, at least a 2.0-fold improvement in solubility within the first 2 hours over a control composition comprising Nilotinib, not being salified with the polymer as measured according to a dissolution profile where the medium is FASSIF having a pH of 6.5.
Aspect 45: The method of any one of aspects 30-44, wherein the composition has an average particle size range from greater than 1 μm to 1000 μm.
Aspect 46: The method of any one of aspects 30-45, wherein the composition has an average particle size range from 1 μm to 20 μm.
Aspect 47: The method of any one of aspects 30-46, wherein the composition does not have an average particle size of less than 1,000 nm.
Aspect 48: The method of any one of aspects 30-47, wherein the control composition is Nilotinib, salified with HCl.
Aspect 49: The method of any one of aspects 30-48, wherein the control composition is a freebase of Nilotinib.
Aspect 50: The method of any one of aspects 30-49, wherein the control composition is Nilotinib, salified with hydrochloride, fumarate, 2-chloromandelate, succinate, adipate, L-tartrate, glutarate, p-toluenesulfonate, camphorsulfonate, glutamate, palmitate, quinate, citrate, maleate, acetate, L-malate, L-aspartate, formate, hydrobromide, oxalate, malonatec, benzenesulfonic acid, butanedisulfonic acid, 1-5-naphthalenedisulfonic acid, naphthalene-1-sulfonic acid, or 1-hydroxynaph-thoic acid.
Aspect 51: The method of any one of aspects 30-50, wherein the composition further comprises a pharmaceutically acceptable carrier or diluent.
Aspect 52: The method of any one of aspects 30-51, wherein the composition further comprises a pharmaceutically acceptable carrier.
Aspect 53: The method of any one of aspects 30-52, wherein the composition is in oral dosage form.
Aspect 54: The method of any one of aspects 30-53, wherein the composition comprises from about 1 mg to about 10 g of the polymer.
Aspect 55: The method of any one of aspects 30-54, wherein the composition comprises from about 20 mg to about 1 g of the polymer.
Aspect 56: The method of any one of aspects 30-55, wherein the composition further comprises one or more excipients.
Aspect 57: The method of any one of aspects 30-56, wherein the composition further comprises one or more excipients selected from the group consisting of colloidal silica, a lubricant, a filler, a disintegrant, a plasticizer, a coloring agent, an emulsifying agent, a diluent, a flavoring agent, a binder, an antioxidant, a light stabilizer, a radical scavenger, a surface active agent, a pH modifier, a drug complexing agent, and a stabilizer against microbial attack, or a combination thereof.
Aspect 58: The method of any one of aspects 30-57, wherein the composition comprises from about 1 mg to about 500 mg of Nilotinib.
Aspect 59: The method of any one of aspects 30-58, wherein the composition comprises from about 20 mg to about 200 mg of Nilotinib.
Aspect 60: The method of any one of aspects 30-59, wherein the composition comprises a weight ratio of Nilotinib:polymer from 20:1 to 1:20.
Aspect 61: The method of any one of aspects 30-60, wherein the composition comprises a weight ratio of Nilotinib:polymer from 20:1 to 1:20.
Aspect 62: The method of any one of aspects 30-61, wherein the composition is an amorphous dispersion.

### Examples

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

The Examples are provided herein to illustrate the invention, and should not be construed as limiting the invention in any way. Examples are provided herein to illustrate the invention and should not be construed as limiting the invention in any way.

### 1. General Experimental Methods

a. Characterization

### (i) Infrared Spectra

Infrared spectra were collected from Nilotinib HPMCP powder samples. A Fourier-transform infrared spectrometer (Tianjin Gangdong Sci. & Tech. Development Co. Ltd.) with an attenuated-total internal reflectance accessory fitted with a diamond crystal was used. The instrument software FTIR-650 spectrometer was used to collect the infrared spectra. Spectra were collected from 4000 to 650 cm⁻¹ for 32 scans at a spectral resolution of 4.0 cm⁻¹. An air background was taken prior to the spectrum of each sample being recorded.

The term “mixture” refers to the fact that compositions of Nilotinib and HPMCP are simple physical mixtures of the type achieved by combining and physically stirring dry components together. Such physical mixtures include wet and dry granulated mixtures. As is known in the art, granulation is a process used to improve the handling and manufacturing properties of a formulation, for example by increasing particle size to improve flow. Granulation does not substantially change the physical form of the drug such as its crystalline or amorphous character. Granulation is not intended to create an amorphous drug/polymer dispersion.

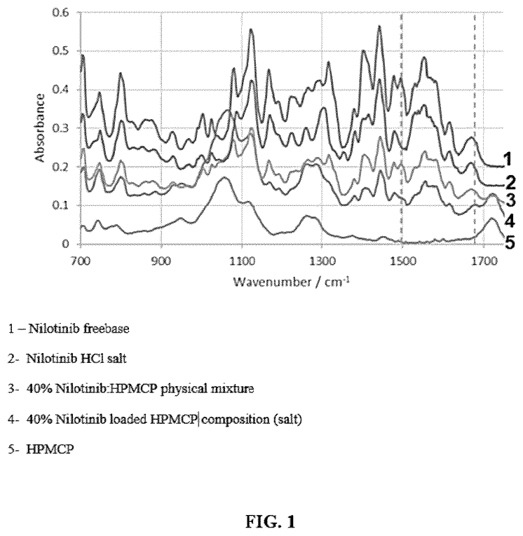

Salt formation between Nilotinib and HPMCP is apparent in the infrared spectrum of a 40% Nilotinib loaded composition made from Nilotinib freebase and HPMCP, see FIG. 1. The physical mixture of Nilotinib freebase and HPMCP and the 40% Nilotinib loaded HPMCP composition infrared spectra are not the same. Thus, if there was no interaction between HPMCP and Nilotinib there would be no difference between these two spectra, see spectra 4 and 3 in FIG. 1. There are spectral differences between the Nilotinib freebase and Nilotinib HCl salt infrared spectra; one of the most important of which is at 1498 cm⁻¹, see spectra 1 and 2 in FIG. 1. This peak is present in the freebase and can be assigned to an N—H deformation mode of the secondary amine, however, in the HCl salt this peak is no longer apparent due to protonation of the secondary amine group. In the physical mixture made from Nilotinib freebase and HPMCP this peak is apparent (see spectra 3 in FIG. 1), however, in the 40% Nilotinib loaded HPMCP composition this peak is not apparent, indicating that the Nilotinib has an ionized secondary amine group. Further, in the 40% Nilotinib loaded HPMCP composition there is a peak at 1691 cm⁻¹; this peak is not coincident with the position of the amide I peak observed in the freebase and Nilotinib HCl salt Nilotinib spectra (which occurs at 1675 cm⁻¹). This peak shifting is a result of the ionization of the carboxylic acid group in the HPMCP. Carboxylic acid salts have a peak in this spectral range; a peak in this range would overlap with the amide I of the Nilotinib and lead to the shift observed. It is anticipated that not all carboxylic acid groups of the HPMCP are deprotonated hence the carboxylic band at 1726 cm⁻¹ is still observed in the 40% Nilotinib loaded composition. Accordingly, FIG. 1 indicates the salt formation between Nilotinib and HPMCP.

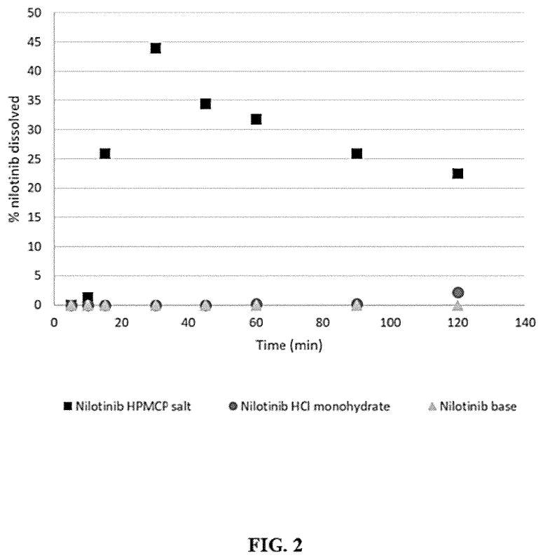

A dissolution profile is shown in FIG. 2 of Nilotinib:HPMCP salt, Nilotinib HCl salt, and Nilotinib free base, where the medium is FaSSIF. In FIG. 2, the dissolution of Nilotinib containing capsule formulations equivalent to 40 mg Nilotinib in 500 mL FaSSIF is shown.

### (ii) Dissolution Tests

For the experiments described herein, a FaSSIF dissolution medium was prepared in the following manner: A stock solution was prepared consisting of 4.44 g of maleic acid, 2.78 g of sodium hydroxide and 8.02 g of sodium chloride in 2 L deionized water adjusted to pH 6.5 using sodium hydroxide. 3.3 g sodium taurocholate was dissolved in 500 mL stock solution. Then 3.15 mL of lecithin dissolved in dichloromethane to 100 mg/mL was added to the sodium taurocholate solution and the dichloromethane was removed applying a vacuum of 250 mbar for 15 min and 100 mbar for 15 min at 40° C. Following cooling of this solution it was diluted to 2 L using stock solution.

A dissolution profile is shown in FIG. 2 of Nilotinib:HPMCP salt, Nilotinib HCl salt, and Nilotinib free base, where the medium is FaSSIF. In FIG. 2, the dissolution of Nilotinib containing capsule formulations equivalent to 40 mg NILOTINIB in 500 mL FaSSIF is shown.

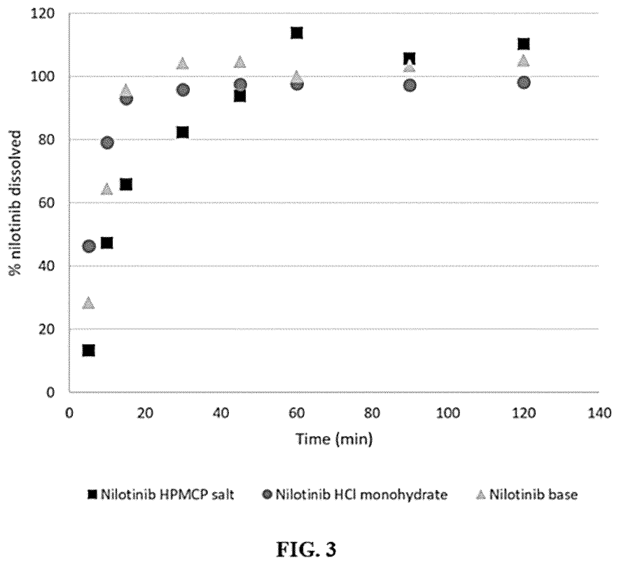

FIG. 3 shows dissolution rate measurement at gastric pH. These were performed using the USP basket method applying stirring at 100 rpm and a use environment consisting of 900 mL of 0.1 M hydrochloric acid pH 1.0, appropriately degassed by vacuum and maintained at a temperature of 37° C. Test or control samples of 40 mg Nilotinib base equivalents were manually filled into size 1 gelatin capsules and placed into baskets before submerging the baskets into the medium. 2 mL liquid was sampled through a cannula at time points 5, 10, 15, 30, 45, 60, 90 and 120 min. Pre-filtration of samples was performed through a paper filter of pore size 2.0 μm into a 5 ml syringe, followed by filtration through a 0.22 μm PES syringe filter. 0.5 mL of filtered sample was immediately diluted with 0.5 mL of acetonitrile into an HPLC vial, and samples were analyzed by HPLC at 266 nm. Test and control samples were measured in duplicate.

FIG. 2 shows dissolution rate measurement at intestinal pH. These were performed using the USP basket method applying stirring at 100 rpm and a use environment consisting of 500 mL of FaSSIF, pH 6.5, appropriately degassed by vacuum and maintained at a temperature of 37° C. Test or control samples of 40 mg Nilotinib base equivalents were manually filled into size 1 gelatin capsules and placed into baskets before submerging the baskets into the medium. 2 mL liquid was sampled through a cannula at time points 5, 10, 15, 30, 45, 60, 90 and 120 min. Pre-filtration of samples was performed through a paper filter of pore size 2.0 μm into a 5 ml syringe, followed by filtration through a 0.22 μm PES syringe filter. 0.5 mL of filtered sample was immediately diluted with 0.5 mL of acetonitrile into an HPLC vial, and samples were analyzed by HPLC at 266 nm. Test samples were measured in duplicate while control samples were not replicated, as these could barely be detected due to low solubility.

(iii) X-Ray Powder Diffraction (XRPD)

XRPD was performed using a PANalytical X'Pert Empyrean system (PW3040/60) (PANalytical B.V., The Netherlands) with CuKα radiation (k=1.542 Å) and a divergence slit of ¼°. The X-ray generator was set to an acceleration voltage of 45 kV and a filament emission of 40 mA. Samples were scanned between 2 and 40° (20) using a step size of 0.01313° and a scan speed of 0.0416°/s. Data were collected using X'Pert Data Collector and viewed using X'Pert Data Viewer (PANalytical B.V., The Netherlands).

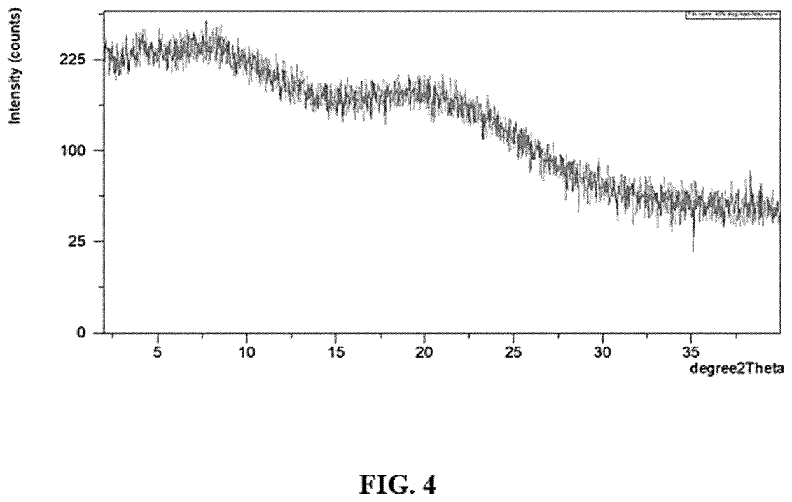

Measurement were performed of physical state and physical stability of the Nilotinib HPMCP salt formulations. FIG. 4 shows the X-ray powder diffractogram of Nilotinib HPMCP salt. The physical state of the Nilotinib HPMCP salt was determined immediately upon isolation using XRPD. The lack of reflections confirmed the amorphous nature of the product. The physical stability over time was tested using Nilotinib HPMCP salt aliquoted in an amount of 50 mg/sample into glass vials and stored without lid in climate chamber at 40° C./75% RH. Sampling for XRPD was performed at time points 7 days, 14 days, 1 month, 2 months and 4 months and diffractograms were obtained using the described XRPD method. The composition remained fully amorphous throughout the study as no reflections were apparent in the diffractograms at any time point.

### (iv) Particle Size Analysis

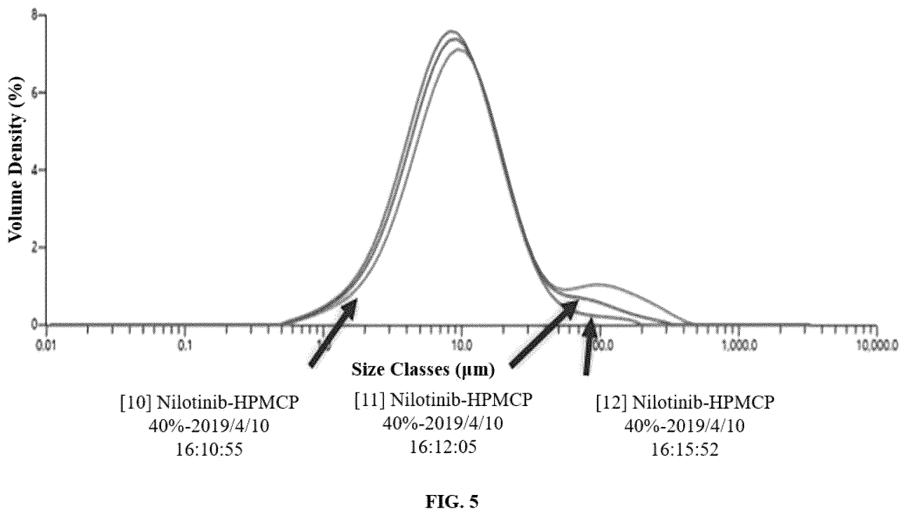

The particle size of the Nilotinib HPMCP salt was determined using a Malvern Mastersizer 3000 equipped with an Aero S dry dispersion unit for dry powder measurement. Approximately 50 mg of sample was placed into the dispersion unit which was operated at a hopper gap of 1.00 mm, a feed rate of 16%, and an air pressure of 3 bar. Laser obscuration was 1.14%. Measurement was performed in triplicate and the result is shown in FIG. 5. The obtained average volume distribution values were as follows: Dv(10) 3.3 μm, Dv(50) 9.1 μm, Dv(90) 35.2 μm. The tail apparent at higher particle diameters in FIG. 5 are likely to represent aggregates of smaller particles.

### (V) Preparation of Nilotinib:HPMCP Salt

A Nilotinib:HPMCP salt was made as follows: a solution of Nilotinib base and HPMCP was made in a mixed solvent of methanol and dichloromethane in a 1:1 v/v ratio allowing ionic bonding between the basic Nilotinib and the HPMCP, which is an acidic polymer, to take place. 1.2 g of HPMCP was dissolved in a volume of the solvent by magnetic stirring, then 0.8 g Nilotinib base was added and allowed to dissolve as well. The solution was transferred to a 100 mL volumetric flask and solvent was added to volume.

The Nilotinib HPMCP salt containing 40% w/w Nilotinib was isolated by spray drying employing a Buchi Mini spray dryer B290 equipped with inert loop B295 (BUCHI Labortechnik AG, Switzerland). A high performance cyclone was used for separation and a 50 ml blue cap flask that could be fitted directly on to the cyclone were used for product collection. Spray dryer process parameters are shown in Table 1.

Following spray drying the product was treated in an oven at 50° C. for 1 hour to evaporate any excess solvent, and the amount of volatiles in the product was determined using TGA. The physical state of the salt was determined immediately using XRPD, see FIG. 4.

TABLE 1

| Parameter | Setting | |
|---|---|---|
| Aspirator | 40 | kg/hour |
| Inlet temp | 85° | C. |
| Outlet temp | 57° | C. |
| Feed rate | 5 | mL/min |
| Atomization airflow | 0.5 | kg/hour |
| Inert loop cooling temp | −20° | C. |

### (VI) In-Vivo Testing

The disclosed compositions can also be tested in vivo in rats as follows: Naïve Sprague Dawley rats (typically n=4-6) that have been fasted the previous day are administered the test or control composition in the fasted or fed state (fasted state: no food is allowed from 12 hours pre-dose until 4 hours post dose).

The test and control compositions are administered, via oral gavage in water or 0.5% HPMC to aid in wetting and prevent caking, through PE205 tubing attached to a syringe. Rats are returned to IVC cages with normal access to water. Alternatively, dosing may be via capsules or tablets. Test and control formulations can be identical except for the presence or absence of polymer. Alternatively, the control formulation can consist of drug alone.

Blood samples are taken from the neck using a 1 ml disposable syringe with a 20 gauge needle at 0.5, 1, 2, 6, 24, and 48 (and occasionally 12 hr.) hours post dose. Other sampling times may be used with the conditions the Tmax is bracketed by the sampling intervals and that an accurate AUC may be calculated. Samples are immediately transferred to clean glass culture tubes containing heparin. Samples are centrifuged at room temperature at 4000 rpm for 10 minutes. Plasma is transferred to clean 1.5 mL Eppendorf tubes using a 51/4″ (13 cm) Pasteur pipette. Plasma samples are stored in a laboratory freezer at −20° C. until assayed by LC/MS.

From plasma or serum drug concentrations, typical pharmacokinetic parameters, such as Cmax, Tmax and AUC can be calculated for each rat, and then averaged for the test population.

Test compositions or controls can be tested in vivo in humans as follows. In a crossover design, 4 or more healthy human subjects are dosed with a suspension of crystalline drug (or amorphous drug if the drug does not crystallize) or a suspension of the drug/polymer composition. Blood samples are taken before dosing and at a variety of times post-dosing, with the number and temporal distribution of sampling times chosen to bracket Tmax and permit accurate measurement of AUC. Drug concentration in plasma or serum is measured by an appropriate assay, and Cmax, Tmax, and AUC can be determined.

The following experiments were performed: Suspensions for oral gavage were prepared containing 15 mg Nilotinib free base as either a 40% w/w drug loaded HPMCP salt or as Nilotinib HCl monohydrate (control). The suspension compositions are presented in Table 2.

TABLE 2

| | | | Marketed Nilotinib | |
|---|---|---|---|---|
| | Nilotinib:HPMCP salt | | salt form | |
| | Nilotinib:HPMCP, 40% | | Nilotinib HCl | |
| Composition | w/w Nilotinib | | monohydrate | |
| Nilotinib free base | 15 | mg | 15 | mg |
| equivalents | | | | |
| Volume of 0.5% HPMC | 6 | mL | 6 | mL |
| solution | | | | |
| Nilotinib | 2.5 | mg/mL | 2.5 | mg/mL |
| suspension conc. | | | | |

After overnight fasting, rats were dosed with a volume of suspension corresponding to 10 mg Nilotinib base/kg body weight, via a gavage tube directly into the stomach. Blood (0.3-0.4 mL using 1 mL syringe and 0.4 diameter needle) was collected from the neck predosing and at 0.5, 1, 2, 6, 24 and 48 hour post-dosing.

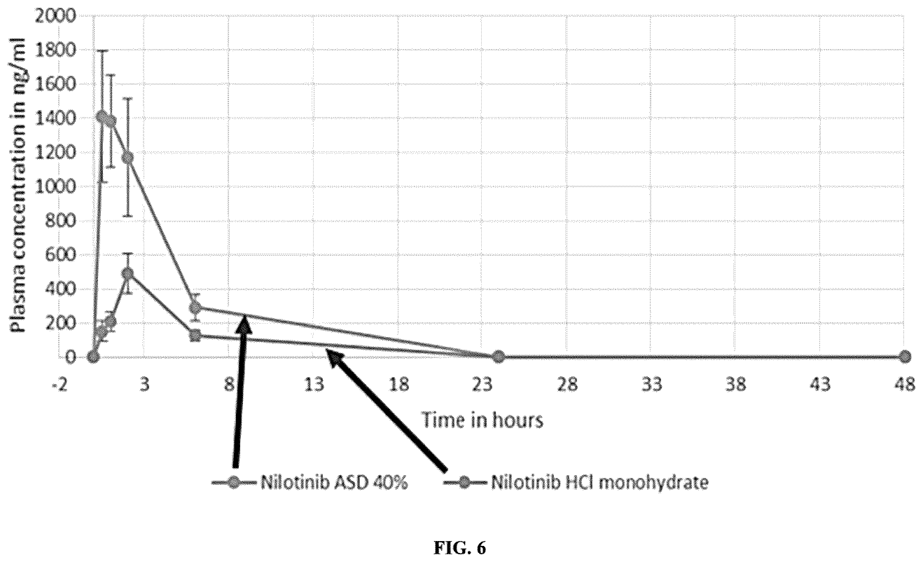

Drug concentrations in plasma were analyzed by LC/MS with ESI in positive ion mode. The ion-pair used for quantification was m/z 539→289. A standard curve was made by mixing 20 μL of standard solutions with 80 μl of blank plasma, and using the above mentioned quantitative analysis. FIG. 6 shows the in-vivo plasma concentration after administrating a single dose equivalent to 10 mg Nilotinib per kg body weight of rats.

100 μL plasma sample was mixed with 400 μL acetonitrile and centrifuged at 12000 RPM for 10 minutes. The supernatant was filtered with a pore size of 0.22 μm.

Pharmacokinetic data are presented in Table 3.

TABLE 3

| Parameter | Nilotinib:HPMCP 2:3 w/w | | Nilotinib HCl monohydrate | |
|---|---|---|---|---|
| AUC (0-t)* | 7902.4 | ng/ml*h | 2911.2 | ng/ml*h |
| F (AUC) | 271.4% | | | |
| Cₘₐₓ | 1491.7 | ng/ml | 492.8 | ng/ml |
| Tₘₐₓ | 0.75 | h | 2.00 | h |

*Linear trapezoidal method

The data shown in Tables 2 and 3 demonstrate that the Nilotinib:HPMCP salt showed a 272% increase in AUC and a 303% increase in Cmax compared to the control when orally dosed to a naïve Sprague Dawley rats.

It will be apparent to those skilled in the art that various modifications and variations can be made in the present invention without departing from the scope or spirit of the invention. Other aspects of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A composition comprising
a) Nilotinib, salified with
b) a polymer selected from the group consisting of hydroxypropylmethyl-cellulose acetate succinate (HPMCAS), hydroxypropylmethylcellulose phthalate (HPMCP), cellulose acetate trimellitate (CAT), cellulose acetate phthalate (CAP), hydroxypropylcellulose acetate phthalate (HPCAP), hydroxypropylmethylcellulose acetate phthalate (HPMCAP), and methylcellulose acetate phthalate (MCAP);
wherein the composition exhibits, in a dissolution experiment, at least a 1.25-fold improvement in solubility within the first 2 hours over a control composition comprising Nilotinib, not being salified with the polymer as measured according to a dissolution profile where FaSSIF is used as a medium and having a pH of 6.5.

2. The composition of claim 1, wherein the Nilotinib is molecularly dispersed in the polymer.

3. The composition of claim 1, wherein the polymer is HPMCP or HPMCAS.

4. The composition of claim 1, wherein the composition exhibits, in a dissolution experiment, at least a 2.0-fold improvement in solubility within the first 2 hours over a control composition comprising Nilotinib, not being salified with the polymer as measured according to a dissolution profile where the medium is FaSSIF having a pH of 6.5.

5. The composition of claim 1, wherein the composition has an average particle size range from greater than about 1 μm to about 1,000 μm.

6. The composition of claim 1, wherein the composition has an average particle size range from 1 μm to 100 μm.

7. The composition of claim 1, wherein the control composition is Nilotinib, salified with HCl.

8. The composition of claim 1, wherein the control composition is a freebase of Nilotinib.

9. The composition of claim 1, wherein the control composition is Nilotinib, salified with hydrochloride, fumarate, 2-chloromandelate, succinate, adipate, L-tartrate, glutarate, p-toluenesulfonate, camphorsulfonate, glutamate, palmitate, quinate, citrate, maleate, acetate, L-malate, L-aspartate, formate, hydrobromide, oxalate, malonatec, benzenesulfonic acid, butanedisulfonic acid, 1-5-naphthalenedisulfonic acid, naphthalene-1-sulfonic acid, or 1-hydroxynaph-thoic acid.

10. The composition of claim 1, wherein the composition further comprises a pharmaceutically acceptable carrier or diluent.

11. The composition of claim 1, wherein the composition is in oral dosage form.

12. The composition of claim 1, wherein the composition comprises from about 20 mg to about 1 g of the polymer.

13. The composition of claim 1, wherein the composition further comprises one or more excipients.

14. The composition of claim 1, wherein the composition comprises from about 1 mg to about 400 mg of Nilotinib.

15. The composition of claim 1, wherein the composition comprises a weight ratio of Nilotinib:polymer from 10:1 to 1:10.

16. The composition of claim 1, wherein the composition is an amorphous dispersion.

17. A method for treating a disorder of uncontrolled cellular proliferation, the method comprising the step of administering to the subject an effective amount of a composition comprising
a) Nilotinib, salified with
b) a polymer selected from the group consisting of hydroxypropylmethyl-cellulose acetate succinate (HPMCAS), hydroxypropylmethylcellulose phthalate (HPMCP), cellulose acetate trimellitate (CAT), cellulose acetate phthalate (CAP), hydroxypropylcellulose acetate phthalate (HPCAP), hydroxypropylmethylcellulose acetate phthalate (HPMCAP), and methylcellulose acetate phthalate (MCAP).

18. The method of claim 17, wherein the disorder of uncontrolled cellular proliferation is a leukemia.

19. The method of claim 17, wherein the subject has been diagnosed with the disorder of uncontrolled cellular proliferation.

20. A composition of Nilotinib salified with HPMCP wherein an infrared spectrum of the composition does not have a N—H deformation peak at 1498 cm⁻¹ and do have a peak at about 1691 cm⁻¹.
